(19) United States
(12) Patent Application Publication
(10) Pub. No.: US 2021/0113492 A1
(43) Pub. Date: Apr. 22, 2021

(54) METHOD FOR INHIBITING PROLIFERATION AND METASTASIS OF CANCER CELLS BY USING NAPHTHOQUINONE DERIVATIVE

(71) Applicant: National Tsing Hua University, Hsinchu (TW)

(72) Inventors: Linyi Chen, Hsinchu (TW); Yu-Jung Chang, Hsinchu (TW); Yen-Chi Tsao, Hsinchu (TW); Chun-Hsien Wang, Hsinchu (TW)

(21) Appl. No.: 16/924,303

(22) Filed: Jul. 9, 2020

Related U.S. Application Data
(60) Provisional application No. 62/924,271, filed on Oct. 22, 2019.

(51) Int. Cl. A61K 31/122; A61P 35/04

(52) U.S. Cl. CPC A61K 31/122; A61P 35/04

(57) ABSTRACT

The present disclosure provides a method for inhibiting proliferation and metastasis of cancer cells by using a naphthoquinone derivative.

### CROSS-REFERENCE TO RELATED APPLICATION

This application claims priority of U.S. patent application No. 62/924,271, filed on Oct. 22, 2019, the content of which is incorporated herein in its entirety by reference.

### BACKGROUND OF THE INVENTION

### 1. Field of the Invention

The present invention relates to a method for inhibiting proliferation and metastasis of cancer cells by using a naphthoquinone derivative.

### 2. The Prior Art

Cancer is one of the main causes of human death today. Although the mechanism of cancer formation is still not fully understood, carcinogenesis or tumorigenesis can be attributed to the accumulation of exogenous or endogenous factors leading to genetic abnormalities in the cells, the signal transduction pathways in these cells would be wrong, causing cell division to be out of control, and then these cells would gradually form cancer cells. Cancer cells can avoid apoptosis and have the ability of migration and invasion. Therefore, cancer cells would continue to proliferate and metastasize to other parts of the body via the lymphatic system or vascular system.

Although there are many drugs currently used in clinical treatment of cancer, the therapeutic effect is still not ideal. The main reasons include: individual differences in patients themselves, serious side effects of anti-cancer drugs, and drug resistance of cancer cells. Therefore, researchers in the art are committed to the development of effective drugs that do not produce undesirable side effects to treat cancer. On the other hand, in addition to generally suppressing the proliferation of cancer cells, suppressing the metastasis of cancer cells is also a very important and difficult problem to be solved.

In order to solve the above problems, those skilled in the art urgently need to develop novel medicaments with the effect of inhibiting the proliferation and metastasis of cancer cells for the benefit of a large group of people in need thereof.

### SUMMARY OF THE INVENTION

A primary objective of the present invention is to provide a method for inhibiting proliferation and metastasis of cancer cells, comprising administering to a subject in need thereof a pharmaceutical composition comprising an effective amount of a naphthoquinone derivative.

According to an embodiment of the present invention, the naphthoquinone derivative is isoplumbagin.

According to an embodiment of the present invention, the effective amount of the isoplumbagin in vitro is at least 1 μM.

According to an embodiment of the present invention, the effective amount of the isoplumbagin in vitro is less than 6 μM.

According to an embodiment of the present invention, the effective amount of the isoplumbagin in vivo is at least 2 mg/kg.

According to an embodiment of the present invention, the cancer cells are oral squamous cell carcinoma cells, human primary glioblastoma cells, human non-small cell lung carcinoma cells, prostate cancer cells, or cervical cancer cells.

According to an embodiment of the present invention, the isoplumbagin is used as a substrate of NAD(P)H quinone dehydrogenase 1 (NQO1).

According to an embodiment of the present invention, the isoplumbagin alters mitochondrial morphogenesis and cellular distribution of cancer cells.

According to an embodiment of the present invention, the isoplumbagin reduces mitochondrial function of cancer cells through inhibiting mitochondrial complex IV activity.

According to an embodiment of the present invention, the isoplumbagin inhibits tumor growth in an oral squamous cell carcinoma xenograft.

In summary, the isoplumbagin of the present invention has the effect on inhibiting proliferation and metastasis of cancer cells (especially oral squamous cell carcinoma cells, human primary glioblastoma cells, human non-small cell lung carcinoma cells, prostate cancer cells, and cervical cancer cells) by targeting NAD(P)H quinone dehydrogenase 1 (NQO1), altering mitochondrial morphogenesis and cellular distribution of cancer cells, and reducing mitochondrial function of cancer cells through inhibiting mitochondrial complex IV activity.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIGS. 1A-1D are schematic diagrams showing the inhibitory effect of isoplumbagin on survival of oral squamous cell carcinoma cells (OC3-IV2), human primary glioblastoma cells (U87), human non-small cell lung carcinoma cells (H1299) and prostate cancer cells (PC3); * indicates P<0.05.

FIGS. 2A-2E show the inhibitory effect of different doses of isoplumbagin on invasion for oral squamous cell carcinoma cells (OC3-IV2), human primary glioblastoma cells (U87), human non-small cell lung carcinoma cells (H1299), prostate cancer cells (PC3) and cervical cancer cells (Hela) assessed with the Boyden chamber assays; * indicates P<0.05.

FIG. 3A identifies candidate target proteins of isoplumbagin. The target prediction of isoplumbagin used target prediction software, Swiss Target Prediction, Pharmmapper, Polypharmacology Browser and Similarity ensemble approach, followed by the Database for Annotation, Visualization and Integrated Discovery (DAVID).

FIG. 3B shows the molecular docking between isoplumbagin and NQO1.

FIG. 4 proves that isoplumbagin is a substrate of NQO1; * indicates P<0.05.

FIGS. 5A-5D show the effect of isoplumbagin on mitochondrial morphogenesis and distribution of oral squamous cell carcinoma cells (OC3-IV2), human primary glioblastoma cells (U87), human non-small cell lung carcinoma cells (H1299) and human prostate cancer cells (PC3). The white square box is the area of the enlarged diagram below, and the alteration of mitochondrial morphogenesis can be more clearly observed.

FIGS. 6A and 6B show the inhibitory effect of isoplumbagin on mitochondrial function and complex IV activity of oral squamous cell carcinoma cells (OC3-IV2); Basal represents basal respiration, ATP represents ATP production ability, Maximal represents maximal respiration, Spare represents spare respiration, and * indicates P<0.05.

FIGS. 7A-7D show the anti-tumor effect of isoplumbagin in xenograft mice models; * indicates P<0.05.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

According to the present invention, the chemical name of isoplumbagin is 5-hydroxy-3-methyl-1,4-naphthoquinone, which has the following chemical formula (I):

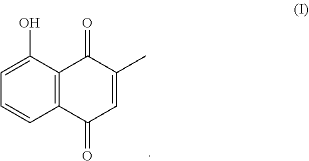

As used herein, the term “treating” or “treatment” refers to reducing, alleviating, ameliorating, relieving, or controlling one or more clinical signs of a disease or disorder, and lowering, stopping, or reversing the progression of severity regarding the condition or symptom being treated.

According to the present invention, the medicament can be manufactured to a dosage form suitable for parenteral or oral administration, using techniques well known to those skilled in the art, including, but not limited to, injection (e.g., sterile aqueous solution or dispersion), sterile powder, tablet, troche, lozenge, pill, capsule, dispersible powder or granule, solution, suspension, emulsion, syrup, elixir, slurry, and the like.

The medicament according to the present invention may be administered by a parenteral route selected from the group consisting of: intraperitoneal injection, subcutaneous injection, intramuscular injection and intravenous injection.

According to the present invention, the medicament may further comprise a pharmaceutically acceptable carrier which is widely used in pharmaceutically manufacturing techniques. For example, the pharmaceutically acceptable carrier can comprise one or more reagents selected from the group consisting of solvent, emulsifier, suspending agent, decomposer, binding agent, excipient, stabilizing agent, chelating agent, diluent, gelling agent, preservative, lubricant, absorption delaying agent, liposome, and the like. The selection and quantity of these reagents fall within the scope of the professional literacy and routine techniques of those skilled in the art.

According to the present invention, the pharmaceutically acceptable carrier comprises a solvent selected from the group consisting of water, normal saline, phosphate buffered saline (PBS), sugar-containing solution, aqueous solution containing alcohol, and combinations thereof.

According to the present invention, the dosage and frequency of administration of isoplumbagin may vary depending on the following factors: the severity of the disease to be treated, the route of administration, and the age, physical condition and response of the individual to be treated. In general, the daily dose of the medicament according to the present invention is usually 0.162 mg/kg body weight, in the form of a single dose or divided into several doses, and can be administered orally or parenterally.

According to the present invention, statistical analysis of results was carried out using the paired Student's t-test or one-way ANOVA. All values reflect the mean ±S.E.M. of data obtained from three independent experiments. Statistical significance was defined as P<0.05.

According to the present invention, the experimental materials and sources used in the following examples are as follows. Isoplumbagin was acquired from AKos GmbH (Steinen, Germany). Anti-TOM20 (#sc-11415) was purchased from Santa Cruz Biotechnology (Santa Cruz, Calif., USA). Alexa Flour 488-conjugated (#A11001) secondary antibodies, Alexa Flour 488-conjugated phalloidin (#A12379) and 4′,6-diamidino-2-phenylindole (DAPI) (#1306) were obtained from Invitrogen (Carlsbad, Calif., USA). Oligomycin (#75351), carbonyl cyanide 4-(trifluoromethoxy)phenylhydrazone (FCCP) (#C2920), rotenone (#R8875), antimycin A (#A8674), lactobionic acid (#153516), taurine (#T8691), digitonin (#D141), glutamate (#G8415), malate (#M1000), succinate (#S3674), adenosine 5′-diphosphate sodium salt (ADP) (#A2754), tetramethyl-p-phenylenediamine (TMPD)(#T3134) and ascorbate (#A4034) were purchased from Sigma-Aldrich (St Louis, Mo., USA).

According to the present invention, human oral cancer cell line OC3-IV2 cells (Lin S C et al, J Oral Pathol Med. 2004 February; 33(2):79-86 and Lu Y C et al, Cancer Prey Res (Phila). 2012 April; 5(4):665-74) were cultured in a 1:1 ratio of Dulbecco's modified Eagle medium (DMEM; Invitrogen) and keratinocyte serum-free medium (KSFM; Invitrogen) containing 10% fetal bovine serum (FBS; Invitrogen, Carlsbad, Calif., USA), 1% L-glutamine (L-Gln, Invitrogen) and 1% antibiotic-antimycotic (AA, Invitrogen). Cells were maintained in a humidified atmosphere containing 5% CO₂ at 37° C., and the culture medium was replaced every 2 days.

According to the present invention, Human primary glioblastoma cell line U87 were obtained from the American Type Culture Collection (ATCC® HTB-14™, Manassas, Va., USA) and grown in DMEM medium supplemented with 10% FBS, 1% MEM Non-Essential Amino Acids Solution (Invitrogen) and 1% penicillin/streptomycin (Invitrogen). The incubator was maintained at 5% CO₂ and a constant temperature of 37° C., and the culture medium was changed every two days.

According to the present invention, human prostate cancer cell line PC3 cells were obtained from ATCC (ATCC® ^{CRL-}1435™) and maintained in DMEM medium supplemented with 10% FBS, 1% L-Gln and 1% AA. Cells were maintained in an atmosphere containing 5% CO₂ at 37° C., and the culture medium was replaced every 2 days.

According to the present invention, human non-small cell lung carcinoma cell H1299 were obtained from ATCC (ATCC® CRL-5803™) cultured in Roswell Park Memorial Institute (RPMI) 1640 medium (Invitrogen) containing 10% FBS, 1% L-Gln and 1% AA. Cells were maintained in a humidified atmosphere containing 5% CO₂ at 37° C., and the culture medium was replaced every 2 days.

According to the present invention, human cervical cancer cell line Hela cells were obtained from ATCC (ATCC® CCL-2™) and maintained in DMEM medium supplemented with 10% FBS, 1% L-Gln and 1% AA. Cells were maintained in an atmosphere containing 5% CO₂ at 37° C., and the culture medium was replaced every 2 days.

### EXAMPLE 1

### Inhibitory Effect of Isoplumbagin on Survival of Oral Squamous Cell Carcinoma Cells (OC3-IV2), Human Primary Glioblastoma Cells (U87), Human Non-small Cell Lung Carcinoma Cells (H1299) and Prostate Cancer Cells (PC3)

This example explores the inhibitory effect of isoplumbagin on survival of oral squamous cell carcinoma cells (OC3-IV2), human primary glioblastoma cells (U87), human non-small cell lung carcinoma cells (H1299) and prostate cancer cells (PC3).

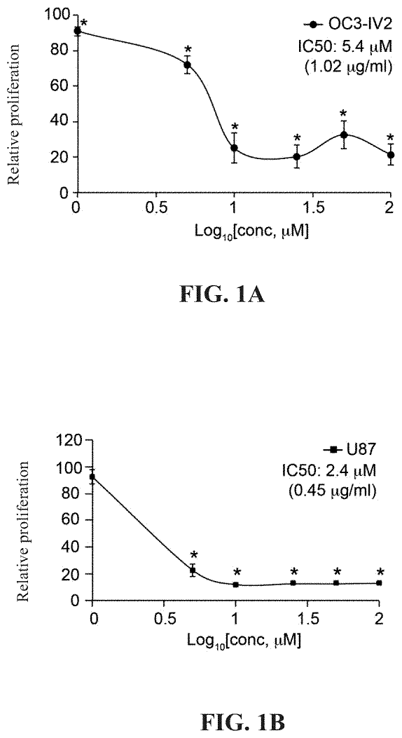

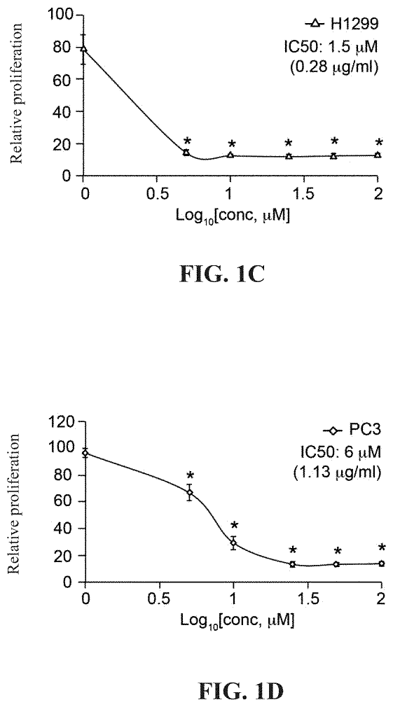

Isoplumbagin (AKOS006277326, acquired from AKos GmbH, Steinen, Germany) was dissolved in dimethyl sulfoxide (DMSO) and added to the medium, so that the final concentration of DMSO was less than 0.1%. The survival rate of isoplumbagin on OC3-IV2, U87, H1299 and PC3 cell lines was determined using the 3-(4,5-dimethylthiazol-2-yl)-2,5-diphenyltetrazolium bromide (MTT; Sigma-Aldrich) assays. 3×10³ cells/well were seeded in 96-well plates. After incubation for 24 h, cells were treated with isoplumbagin at various concentrations (0, 1, 5, 10, 25, 50 and 100 μM) and incubated for a further 48 h. At the end of treatment, 10 μL 5 mg/mL MTT was added to each well. After incubation at 37° C. for 3 h, medium was removed and DMSO was added to each well to dissolve reduced MTT product formazan. The absorbance of dissolved formazan was measured at 550 nm wavelengths by a spectrophotometer. The experimental results are shown in FIGS. 1A-1D.

FIGS. 1A-1D are schematic diagrams showing the inhibitory effect of isoplumbagin on survival of oral squamous cell carcinoma cells (OC3-IV2), human primary glioblastoma cells (U87), human non-small cell lung carcinoma cells (H1299) and prostate cancer cells (PC3). The MTT assay was used to determine survival rate of OC3-IV2 (A), U87 (B), H1299 (C) and PC3 (D) cells treated with isoplumbagin for 48 h. *Compared with solvent control (DMSO). Data from three independent experiments are presented as mean ±SEM (*P<0.05, paired Student's t-test). The result of this example shows that isoplumbagin can effectively inhibit the survival rate of oral squamous cell carcinoma cells (OC3-IV2)(IC50 of isoplumbagin was 1.02 μg/mL), human primary glioblastoma cells (U87)(IC50 of isoplumbagin was 0.45 μg/mL), human non-small cell lung carcinoma cells (H1299)(IC50 of isoplumbagin was 0.28 μg/mL) and prostate cancer cells (PC3)(IC50 of isoplumbagin was 1.13 μg/mL).

### EXAMPLE 2

Inhibitory Effect of Different Doses of Isoplumbagin on Invasion for Oral Squamous Cell Carcinoma Cells (OC3-IV2), Human Primary Glioblastoma Cells (U87), Human Non-small Cell Lung Carcinoma Cells (H1299), Prostate Cancer Cells (PC3) and Cervical Cancer Cells (Hela) Assessed with Boyden Chamber Assays

In this example, the inhibitory effect of isoplumbagin on invasion for oral squamous cell carcinoma cells (OC3-IV2), human primary glioblastoma cells (U87), human non-small cell lung carcinoma cells (H1299), human prostate cancer cells (PC3) and human cervical cancer cells (Hela) was observed using the Boyden chamber assays.

The source and culture method of oral squamous cell carcinoma cells (OC3-IV2), human primary glioblastoma cells (U87), human prostate cancer cells (PC3), human non-small cell lung carcinoma cells (H1299) and human cervical cancer cells (Hela) were the same as those described above.

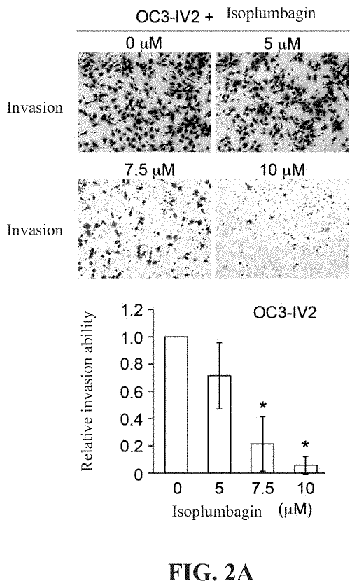

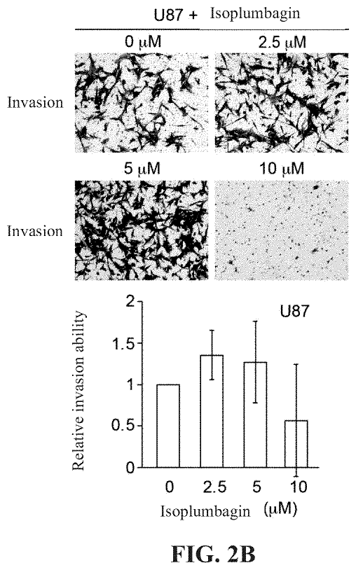

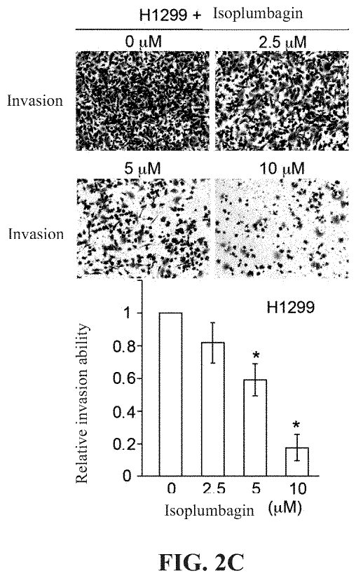

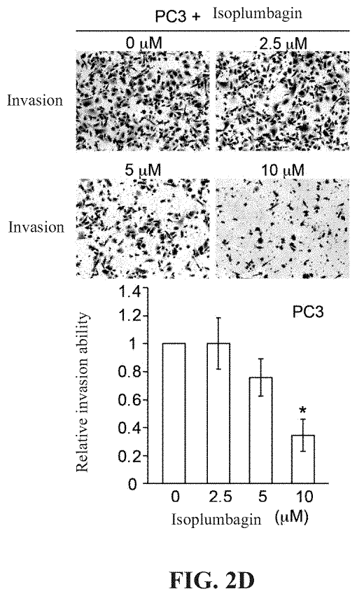

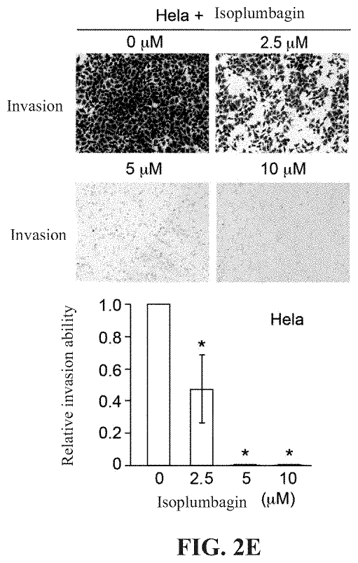

Cells were harvested by trypan and resuspended in a serum-free medium with 0.1% BSA. Then, 200 ml of cell suspension (2×10⁵ cells) was seeded into the upper insert of SPLInsert with Polyethylene terephthalate (PET) membrane (pore size: 8.0 μm) (SPL Lifesciences, Korea) pre-coated with matrigel (BD Biosciences, San Jose, Calif.). The bottom well of 24-well plate of SPLInsert was filled with cell medium containing 10% FBS with different concentrations of isoplumbagin (5, 7.5 and 10 μM, Experimental group of oral squamous cell carcinoma cells (OC3-IV2); 2.5, 5 and 10 μM, Experimental groups of human primary glioblastoma cells (U87), human non-small cell lung carcinoma cells (H1299), human prostate cancer cells (PC3) and human cervical cancer cells (Hela)) or without isoplumbagin (DMSO added). DMSO or different concentrations of isoplumbagin (5, 7.5 and 10 μM, Experimental group of oral squamous cell carcinoma cells (OC3-IV2); 2.5, 5 and 10 μM, Experimental groups of human primary glioblastoma cells (U87), human non-small cell lung carcinoma cells (H1299), human prostate cancer cells (PC3) and human cervical cancer cells (Hela)) were separately added in the suspension of cells in the upper insert. After incubation for 24 h, the cells migrated to the bottom side of PET membrane were fixed with 4% paraformaldehyde and stained with crystal violet. Photos of stained cells on the underside of PET membrane were taken using Zeiss Observer Z1 microscope (Zeiss, Jena, Germany). Number of cells was counted using Image J. Relative invasion ability was calculated as cell number per area. The experimental results are shown in FIGS. 2A-2E.

FIGS. 2A-2E show the inhibitory effect of different doses of isoplumbagin on invasion for oral squamous cell carcinoma cells (OC3-IV2), human primary glioblastoma cells (U87), human non-small cell lung carcinoma cells (H1299), prostate cancer cells (PC3) and cervical cancer cells (Hela) assessed with the Boyden chamber assays. *Compared with solvent control (DMSO). Data from three independent experiments are presented as mean ±SEM (*P<0.05, paired Student's t-test). The results of FIGS. 2A to 2E show that the cancer cells migrating through the polycarbonate membrane decrease significantly as the dose increases. Among them, there is a dose dependency in FIGS. 2A and 2C-2E, while there is no dose dependency in FIG. 2B. The result of this example confirms that isoplumbagin has a good effect on inhibiting cancer cells, especially in the ability to inhibit the invasion of cancer cells.

### EXAMPLE 3

### Identification of Candidate Target Proteins of Isoplumbagin and Confirmation of Isoplumbagin as a Substrate of NQO1

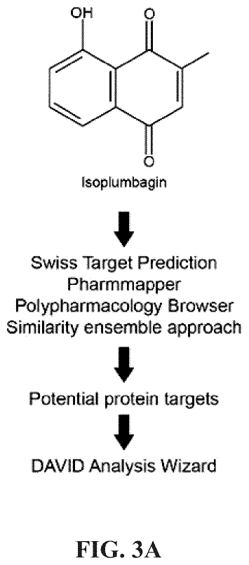

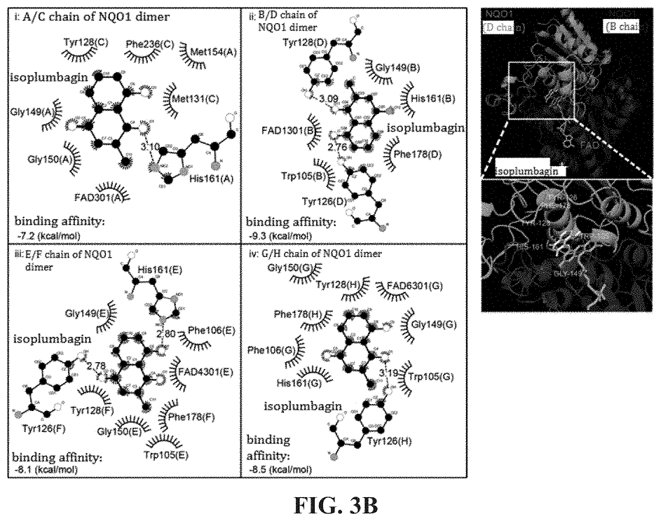

In this example, target prediction softwares, Swiss Target Prediction (SIB Swiss Institute of Bioinformatics, Lausanne, Switzerland), Pharmmapper (Xiaofeng Liu et al, Nucleic Acids Res. 2010 July; 38(Web Server issue):W609-14.), Polypharmacology Browser (Awale M et al, J Cheminform 2017 Feb. 21; 9:11.) and Similarity ensemble approach (Keiser M J et al, Nat Biotechnol. 2007 February; 25(2):197-206.), followed by the Database for Annotation, Visualization and Integrated Discovery (DAVID) were used to identify candidate target proteins of isoplumbagin and confirm isoplumbagin is a substrate of NAD(P)H quinone dehydrogenase 1 (NQO1). To perform molecular docking of NQO1 dimer and isoplumbagin, the crystallographic structure of NQO1 (PDB code 2F1O, comprising four NQO1 dimers (A/C chain dimer, B/D chain dimer, E/F chain dimer, G/H chain dimer) and isoplumbagin (CID: 375105) was applied. The complex model of NQO1 and isoplumbagin was performed using PyRx (Dallakyan et al, Methods Mol Biol. 2015; 1263:243-50.). The structural model representations and docked orientations were generated by PyMOL (DeLano Scientific) and Ligplot (Laskowski et al, J Chem Inf Model. 2011 Oct. 24; 51(10):2778-86.). The experimental results are shown in Table 1, FIG. 3A and FIG. 3B. The ES in Table 1 is Enrichment Score. The enrichment score (ES) is the maximum deviation from zero encountered during that walk. The ES reflects the degree to which the genes in a gene set are overrepresented at the top or bottom of the entire ranked list of genes.

TABLE 1

| Term | Count | ES | P-value | Gene |
|---|---|---|---|---|
| Oxidation- | 16 | 5.35 | 1.4E−5 | NQO1, AKR1C3, ALOX15, |
| reduction | | | | DCXR, DHODH, DBH, GSR, |
| process | | | | HSD17B7, IDO1, IMPDH2, |
| | | | | KDM4E, MAOA, MAOB, |
| | | | | NOS2, NOS3, TDO2 |
| Signal | 14 | — | 5.7E−2 | JAK2, CASP1, DAPPK1, |
| trans- | | | | ESR1, FGG, HINT1, ITPKA, |
| duction | | | | MAPK10, MAPK14, NR1l2, |
| | | | | PDE4B, PDE4D, PGR, |
| | | | | STAT3 |
| Response | 11 | — | 4.9E−5 | ABL1, ADAM17, LCK, |
| to drug | | | | DHODH, DUSP6, HADH, |
| | | | | MAOB, PTEN, STAT1, |
| | | | | STAT3, TOP1 |
| Negative | 11 | — | 1.2E−3 | MALT1, NQO1, ALOX12, |
| regulation | | | | DUSP1, GSTP1, GSK3B, |
| of apoptosis | | | | MAPK8, PTEN, RARG, |
| process | | | | STAT3, TP53 |
| Protein | 11 | 4.37 | 1.2E−3 | BTK, JAK2, LCK, CDC25B, |
| phosphory- | | | | CDK6, DAPK1, GSK38, |
| lation | | | | ITPKA, MAPK10, MAPK8, |
| | | | | PDK2 |

Table 1 is the Gene Ontology (GO) term enrichment results of potential binding targets for isoplumbagin based on DAVID analysis. FIG. 3A identifies candidate target proteins of isoplumbagin. The target prediction of isoplumbagin used target prediction softwares, Swiss Target Prediction, Pharmmapper, Polypharmacology Browser and Similarity ensemble approach, followed by the Database for Annotation, Visualization and Integrated Discovery (DAVID). FIG. 3B shows the molecular docking between isoplumbagin and NQO1 dimers (A/C chain dimer, B/D chain dimer, E/F chain dimer, G/H chain dimer).

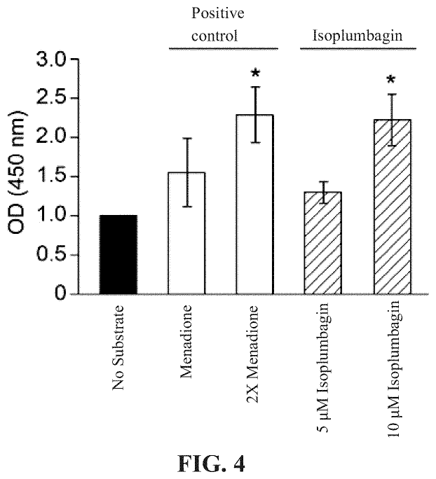

FIG. 4 proves that isoplumbagin is a substrate of NQO1. The cytosolic fractions of OC3-IV2 cells were used for the NQO1 enzymatic activity assay. The NQO1 enzyme activity was calculated by measuring the simultaneous reduction of menadione or isoplumbagin with cofactor NADH and WST1 (a highly sensitive tetrazolium reagent) which leads to increased absorbance at 450 nm. NQO1 activity assay kit (#ab184867) was purchased from Abcam (Cambridge, Mass., USA). OC3-IV2 cells were lysed with 1X extraction buffer containing 1 mM phenylmethylsulfonyl fluoride (PMSF) for 15 min on ice, and then centrifuged at 17,000×g at 4° C. for 20 min. The supernatant was collected and then the protein concentration of each sample was determined by BCA assays (Santa Cruz Biotechnology). Diluted sample and reaction buffer were added into 96-wells plate containing menadione (1X and 2X) or different concentrations of isoplumbagin (5 μM and 10 μM) with cofactor NADH and WST1. The absorbance of reduced WST1 was measured at 450 nm wavelengths by a spectrophotometer. Data from three independent experiments are presented as mean ±SEM (*P<0.05 compared with no substrate, paired Student's t-test). Therefore, it is confirmed that isoplumbagin and menadione are substrates of NQO1.

### EXAMPLE 4

### Effect of Isoplumbagin on Mitochondrial Morphogenesis and Distribution of Oral Squamous Cell Carcinoma Cells (OC3-IV2), Human Primary Glioblastoma Cells (U87), Human Non-small Cell Lung Carcinoma Cells (H1299) and Human Prostate Cancer Cells (PC3)

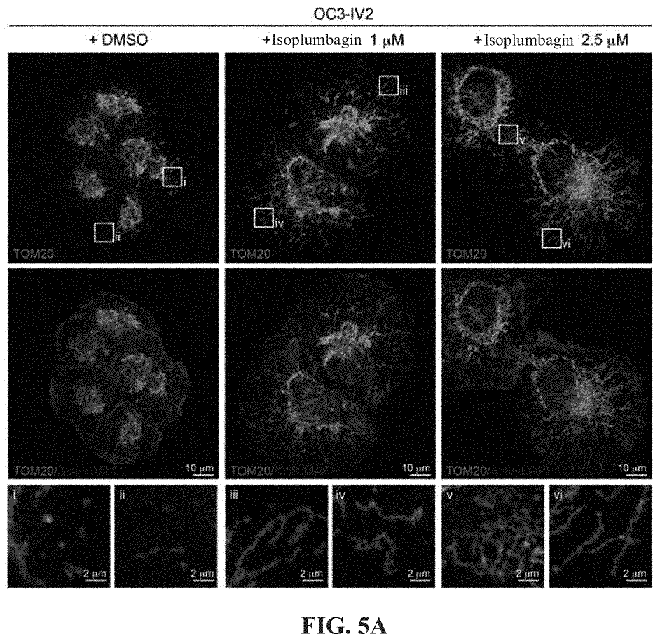

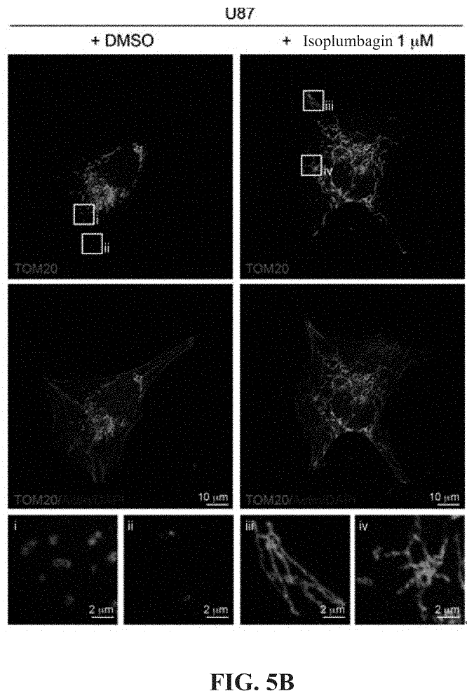

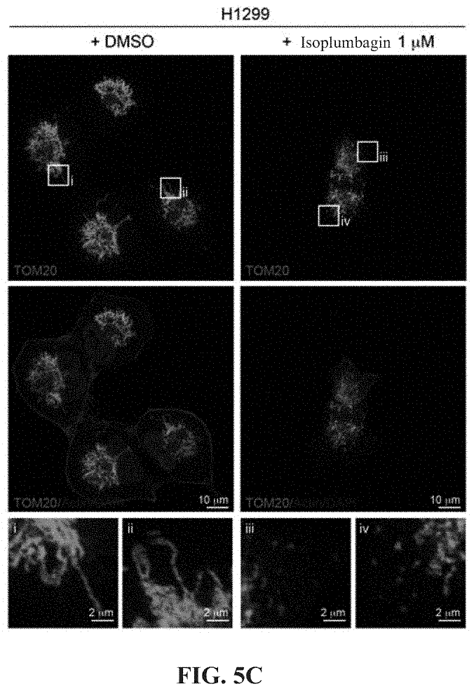

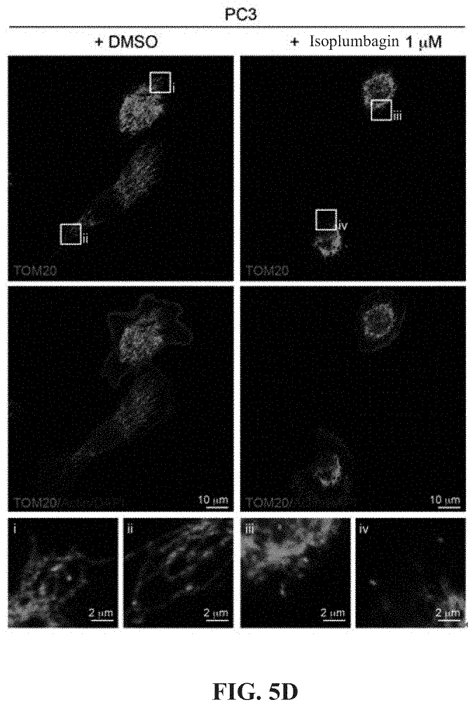

The effects of isoplumbagin on mitochondrial morphology and distribution of OC3-IV2, U87, H1299 and PC3 cell lines were measured by immunofluorescence staining After OC3-IV2, U87, H1299, and PC3 cells were treated with DMSO, 1 μM, or 2.5 μM isoplumbagin for 24 hours, cells were fixed by 4% (v/v) paraformaldehyde and permeabilized by 0.1% (v/v) Triton X-100. Cells were incubated in blocking buffer containing 1% bovine serum albumin. After blocking, cells were incubated overnight with indicated primary antibodies against TOM20 (a mitochondria marker, sc-11415, Santa Cruz), and then incubated with Alexa Fluor 488-conjugated secondary antibodies (A11001, Invitrogen). Nucleus was stained with DAPI, actin was stained with rhodamine phalloidin (A12379, Invitrogen) and cells were mounted using Prolong Gold reagent (P36930, Invitrogen). 63 images were taken by LSM800 confocal microscopes (Zeiss). The results are shown in FIGS. 5A-5D.

FIGS. 5A-5D show the effect of isoplumbagin on mitochondrial morphogenesis and distribution of oral squamous cell carcinoma cells (OC3-IV2), human primary glioblastoma cells (U87), human non-small cell lung carcinoma cells (H1299) and human prostate cancer cells (PC3). Cells treated with either solvent control (DMSO) or isoplumbagin were determined by immunofluorescence staining using anti-TOM20 (mitochondria, green) antibody, rhodamine phalloidin (actin, red) and DAPI (nucleus, blue). Enlarged panels from the square box area are shown in the bottom panel. The results of this example show that isoplumbagin can alter the mitochondrial morphogenesis and distribution of cancer cells, indicating that the cells are affected by the cytotoxic effect of isoplumbagin to produce cell stress response, which promotes the alterations of mitochondrial morphogenesis and cell distribution (Chien L et al, Oncotarget. 2015 Oct. 13; 6(31):30628-39; Chien L et al, Biochim Biophys Acta Mol Basis Dis. 2018 September; 1864(9 Pt B):3001-3012.).

### EXAMPLE 5

### Inhibitory Effect of Isoplumbagin on Mitochondrial Function and Complex IV Activity of Oral Squamous Cell Carcinoma Cells (OC3-IV2)

In this example, the inhibitory effect of isoplumbagin on mitochondrial function and complex IV activity of oral squamous cell carcinoma cells (OC3-IV2) was explored.

Oxygen consumption rate (OCR) in intact cells suspension was measured by O2k Oxygraph (Oroboros Instruments). DMSO or 2.5 μM isoplumbagin treated cells were detached from the plate by trypsinization and suspended at 5×10⁵ cells/ml in DMEM/KSFM medium supplemented with 10% FBS into the O2k chambers. Oxygen consumption rate of DMSO or isoplumbagin treated cells samples were measured by adding 0.5 μM oligomycin, 0.5 μM FCCP, 1 μM rotenone and 1 μM antimycin A.

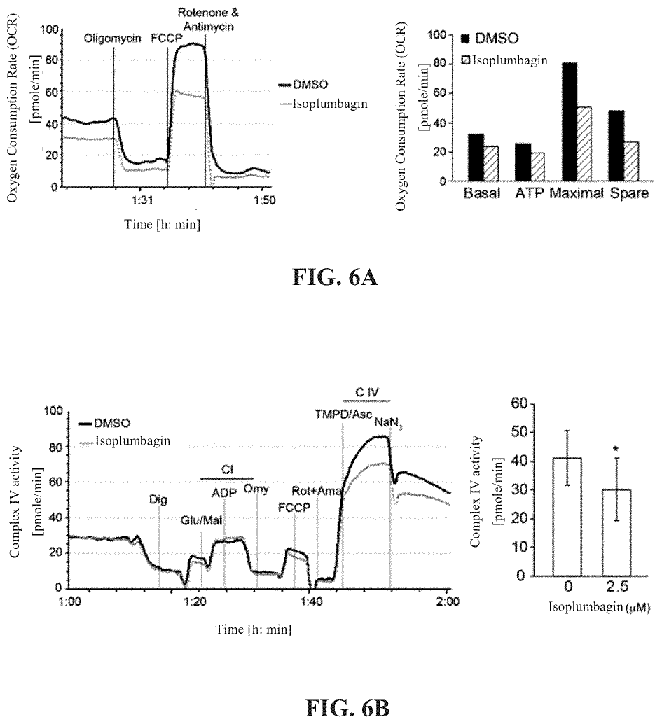

For the cell permeabilization and measurement of mitochondrial complex activity in permeabilized cells using Oroboros Instruments, 5×10⁵ cells/mL treated with DMSO or 2.5 μM isoplumbagin suspended in 2 mL MiR05 buffer (110 mM D-sucrose, 0.5 mM EGTA, 3.0 mM MgCl₂, 60 mM lactobionic acid, 10 mM KH₂PO₄, 20 mM taurine, 20 mM HEPES, 1 g/L BSA, pH 7.1) were added in O2k Oxygraph chambers. After cells had stabilized at routine respiration, plasma membrane permeabilization was performed by adding 5 μM digitonin and then O₂ consumption rate was measured in response to sequential additions of 10 mM glutamate and 2 mM malate or 10 mM succinate, followed by 5 mM ADP, 0.5 mM oligomycin, 0.5 μM FCCP, 1 μM rotenone, 1 μM antimycin A, 0.1 mM TMPD, 0.4 mM ascorbate and 5 mM NaN₃. The results are shown in FIGS. 6A and 6B.

FIGS. 6A and 6B show the inhibitory effect of isoplumbagin on mitochondrial function and complex IV activity of oral squamous cell carcinoma cells (OC3-IV2). Oxygen consumption rate (OCR) of OC3-IV2 cells treated with DMSO (as control) or 2.5 μM isoplumbagin were measured by Oroboros instruments (FIG. 6A). This result was from one independent experiment. OC3-IV2 cells treated with DMSO or 2.5 μM isoplumbagin were permeabilized with 5 μM digitonin (Dig) and added indicated substrates and inhibitors, and then mitochondria complex I/IV activity were determined by Oroboros instruments (FIG. 6B). Glu/Mal: glutamate (10 mM) and malate (2 mM), ADP (5 mM), Omy: oligomycin (0.5 μM), FCCP (0.5 nM), Rot: rotenone (1 μM), Ama: antimycin A (2 μM), TMPD: tetramethyl-p-phenylenediamine (0.1 mM), Asc: ascorbate (0.4 mM) and NaN₃ 5 mM. Data from three independent experiments are presented as mean ±SEM (*P<0.05, paired Student's t-test). The result of this example indicates that isoplumbagin can reduce mitochondrial function of cancer cells through inhibiting mitochondrial complex IV activity.

### EXAMPLE 6

### Anti-tumor Effect of Isoplumbagin in Xenograft Mice Model

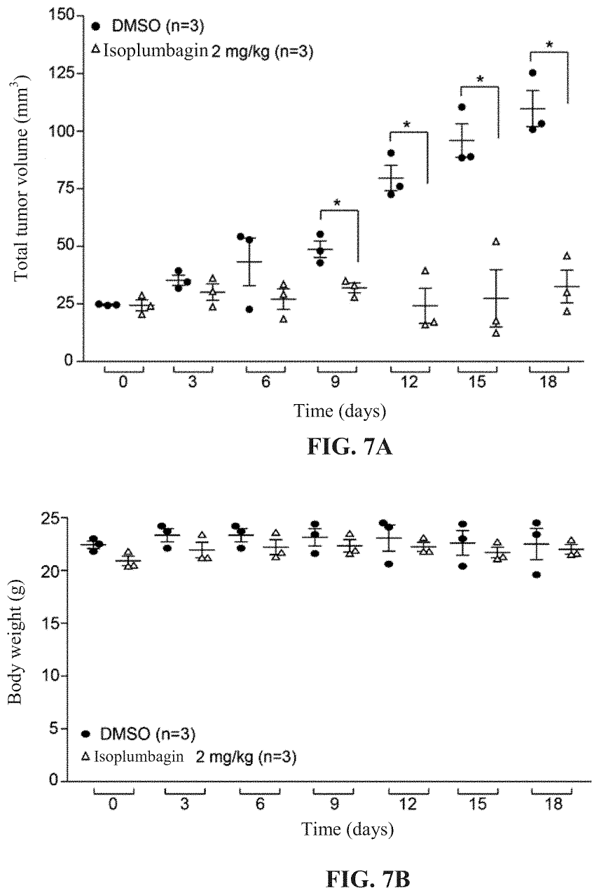

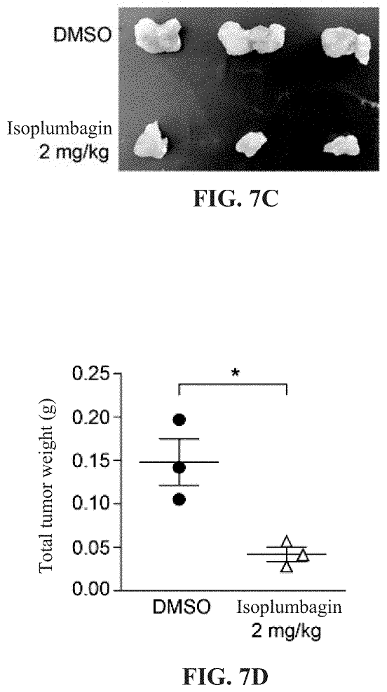

In this example, the anti-tumor effect of isoplumbagin in xenograft mice model was explored. In oral orthotopic xenograft CB17/lcr-Prkdc^{scid}/CrlNarl mice model, 1×10⁶ OC3-IV2 cells were harvested and re-suspended in 100 μl PBS. The cells were injected through the oral buccal mucosa into six-week-old male CB17/lcr-Prkdc^{scid}/CrlNarl mice (National Laboratory Animal Center, Taiwan). When the tumor size in oral buccal reached approximately 25 mm³ at 14 days after inoculation of the OC3-IV2 cells, mice were randomly assigned into two groups (n=3 per group): vehicle (DMSO) control group and 2 mg/kg isoplumbagin group. Treatments were given via intraperitoneal injection once every 3 days in the morning for 2 weeks. During this period, mice weights and tumor volumes (length×width²/2) were recorded every 3 days. At the end of the study (on day 18), the mice were sacrificed by carbon dioxide asphyxiation. All procedures were performed according to approved National Tsing Hua University Institutional Animal Care and Use Committee (approval number: 10602, approval date: 2017 Feb. 17) and National Chiao Tung University Institutional Animal Care and Use Committee (approval number NCTU-IACUC-108032, approval date: 2019 Jul. 22) protocols. The results are shown in FIGS. 7A-7D.

FIGS. 7A-7D show the anti-tumor effect of isoplumbagin in xenograft mice model. FIG. 7A shows OC3-IV2 cells were injected into the oral buccal mucosa of CB17/lcr-Prkdc^{scid}/CrlNarl mice. After tumor volume reached approximately 25 mm³, mice were treated with vehicle (DMSO, n=3) or isoplumbagin (2 mg/kg, n=3) via intraperitoneal injections once every 3 days. Tumor volume (FIG. 7A) and body weight (FIG. 7B) were measured from day 0-18 after treatment. FIG. 7C shows mice were sacrificed and tumors were isolated from the mice at the end of treatment at 18 days. Photographs of tumors are shown. FIG. 7D shows average tumor weight of harvested tumor from FIG. 7C. Each value in the graph represents the mean ±SEM from three mice (*P<0.05, one-way ANOVA). The results of this example indicate that isoplumbagin can effectively inhibit tumor growth in vitro in oral squamous cell carcinoma xenograft model.

In summary, the isoplumbagin of the present invention has the effect on inhibiting proliferation and metastasis of cancer cells (especially oral squamous cell carcinoma cells, human primary glioblastoma cells, human non-small cell lung carcinoma cells, prostate cancer cells, and cervical cancer cells) by targeting NAD(P)H quinone dehydrogenase 1 (NQO1), altering mitochondrial morphogenesis and cellular distribution of cancer cells, and reducing mitochondrial function of cancer cells through inhibiting mitochondrial complex IV activity.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for inhibiting proliferation and metastasis of cancer cells, comprising administering to a subject in need thereof a pharmaceutical composition comprising an effective amount of a naphthoquinone derivative.

2. The method according to claim 1, wherein the naphthoquinone derivative is isoplumbagin.

3. The method according to claim 2, wherein the effective amount of the isoplumbagin in vitro is at least 1 μM.

4. The method according to claim 3, wherein the effective amount of the isoplumbagin in vitro is less than 6 μM.

5. The method according to claim 2, wherein the effective amount of the isoplumbagin in vivo is at least 2 mg/kg.

6. The method according to claim 1, wherein the cancer cells are oral squamous cell carcinoma cells, human primary glioblastoma cells, human non-small cell lung carcinoma cells, prostate cancer cells, or cervical cancer cells.

7. The method according to claim 2, wherein the isoplumbagin is used as a substrate of NAD(P)H quinone dehydrogenase 1 (NQO1).

8. The method according to claim 2, wherein the isoplumbagin alters mitochondrial morphogenesis and cellular distribution of cancer cells.

9. The method according to claim 8, wherein the isoplumbagin reduces mitochondrial function of cancer cells through inhibiting mitochondrial complex IV activity.

10. The method according to claim 6, wherein the isoplumbagin inhibits tumor growth in an oral squamous cell carcinoma xenograft.
